(19) United States
(12) Patent Application Publication
(10) Pub. No.: US 2012/0259405 A1
(43) Pub. Date: Oct. 11, 2012

(54) Radial Radiopaque Markers

(75) Inventors: Jan Weber, Maastricht (NL); Aiden Flanagan, Galway (IE)

(73) Assignee: BOSTON SCIENTIFIC SCIMED, INC, Maple Grove, MN (US)

(21) Appl. No.: 13/431,454

(22) Filed: Mar. 27, 2012

Related U.S. Application Data
(60) Provisional application No. 61/471,898, filed on Apr. 5, 2011.

(51) Int. Cl. A61F 2/82; A61M 29/02

(52) U.S. Cl. 623/1.15; 606/192

(57) ABSTRACT

A deployable medical device having at least one radiopaque marker is herein disclosed. In particular, the deployable medical device comprises a bio-absorbable stent or expandable medical balloon. Upon insertion of the deployable medical device, the at least one radiopaque marker is inserted into adjacent tissue, where it will remain even after the deployable medical device has been removed.

### CROSS-REFERENCE TO RELATED APPLICATIONS

This application claims priority to U.S. provisional application Ser. No. 61/471,898, filed Apr. 5, 2011, the contents of which are hereby incorporated by reference.

### STATEMENT REGARDING FEDERALLY SPONSORED RESEARCH

Not Applicable.

### FIELD OF THE INVENTION

This invention relates to radiopaque markers, and more specifically to radiopaque markers that can be used in conjunction with stents, medical balloons, and other implantable medical devices.

### BACKGROUND OF THE INVENTION

Various types of bio-absorbable stents are known. One of the problems associated with bio-absorbable stents, however, is that they do not show up well under fluoroscopy. For example, bio-absorbable stents comprising magnesium, or stents made of polymeric material, are not particularly radiopaque. To increase the radiopacity of certain products made out these materials having low inherent opacity, radiopaque markers or coatings have heretofore been added to the stent. Use of radiopaque markers, however, is problematic due to the bio-absorbable material of the stent. In particular, as the stent is absorbed, the radiopaque markers are no longer restrained by the stent. As a result, the radiopaque markers, which can be tens of micrometers in size, are permitted to drift downstream, possibly constricting blood flow.

Radiopaque coatings suffer from essentially the same problem. Radiopaque coatings of gold, for example, are required to be approximately five micrometers in thickness to be sufficiently visible under fluoroscopy. Upon degradation and absorption of the underlying stent, the remaining radiopaque film coating can create risks of constricting blood flow or even thrombosis.

Using radiopaque coatings on a bio-absorbable metallic stent can also be problematic. Sufficient radiopacity is obtained using heavy elements, for example, gold, platinum, tantalum, tungsten, iridium, ruthenium, and the like. And, even with these materials, the radiopaque coatings need to be 5 micrometers in thickness to supply the desired level of radiopacity. A metallic coating different from the underlying material of the bio-absorbable metallic stent, however, produces undesirable micro galvanic cells.

Another problem associated with radiopaque markers or coatings applied to bio-absorbable stents is that when the stent is absorbed, the markers or coatings can no longer be used to identify the area being stented. In this respect, it is desirable to have a permanent marker that identifies the area or areas being stented even after the stents have been absorbed. Similarly, it is desirable to identify areas which have been subjected to medical balloon procedures, including medical balloons that have been treated with drugs, well after the procedure is complete and the balloon has been removed. In particular, it is useful to know the location of previously treated areas in order to avoid double treatment, particularly in the timeframe in which the initial treatment is still of influence.

Consequently, there remains a need for permanent radiopaque markers that can be used in conjunction with balloons, stents, including bio-absorbable stents, and other implantable medical devices.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

In some embodiments, an implantable medical device comprises a bio-absorbable stent and at least one radiopaque anchor. The at least one radiopaque anchor extends radially from the bio-absorbable stent and is attached thereto. In addition, the implantable medical device comprises a biocompatible polymer that contacts at least a portion of the bio-absorbable stent and at least a portion of the at least one radiopaque anchor.

In some embodiments, a medical device comprises a medical balloon. The medical balloon comprises an unexpanded configuration and an expanded configuration. In addition, the medical balloon comprises at least one radiopaque anchor that extends radially from the medical balloon when the medical balloon is in the expanded configuration. Finally, the at least one radiopaque anchor is detachably fastened to the medical balloon.

### BRIEF DESCRIPTION OF THE SEVERAL VIEWS OF THE DRAWING(S)

FIG. 1 shows a perspective view of an embodiment of a deployable medical device, in the form of a stent, having radiopaque anchors.

FIGS. 2A-2D show cross-sectional views of valleys 14 of the stent of FIG. 1.

FIGS. 3A-3G show side views of the radiopaque anchor.

FIG. 4 shows a side view of an embodiment of a deployable medical device, in the form of an expandable balloon, having radiopaque anchors.

FIG. 4A shows a cross-sectional view of a portion of the expandable balloon of FIG. 4.

FIG. 5 shows a cross-sectional view of an expandable balloon in a folded configuration.

FIG. 5A shows a cross-sectional view of the expandable balloon of FIG. 5 in an expanded configuration.

### DETAILED DESCRIPTION OF THE INVENTION

In at least one embodiment, a deployable medical device 10 comprises a radiopaque anchor 20 located along an outer surface of the deployable medical device 10. In some embodiments, the radiopaque anchor 20 extends radially outwardly from the deployable medical device 10. As used herein, the term “anchor” refers to a device that is implantable within the tissue of a bodily structure, for example the wall of a blood vessel. Moreover, as used herein with respect to the materials from which components or portions of the immediate device is manufactured, the term “permanent” means that the material is intended to last for the life of the patient without significant decay.

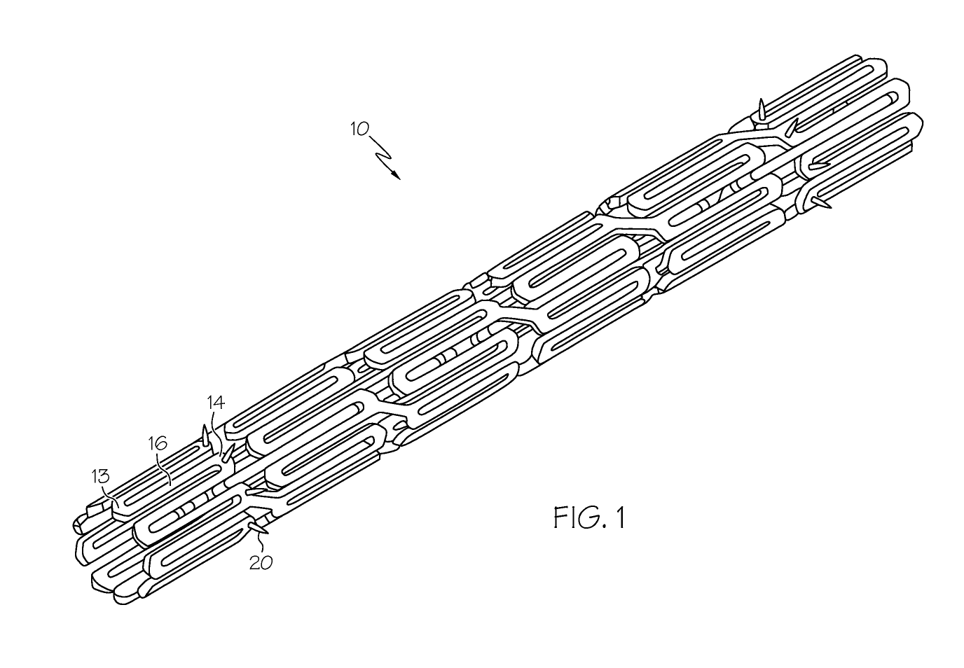

In some embodiments, for example as shown in FIG. 1, the deployable medical device 10 comprises a stent 12. For the purposes of illustration, in some embodiments, the stent 12 comprises a plurality of struts 16, which are interconnected at peaks 13 and valleys 14.

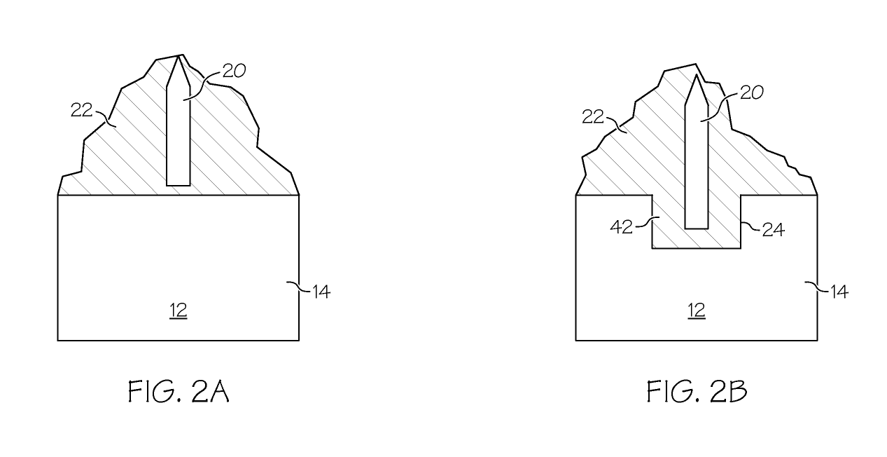

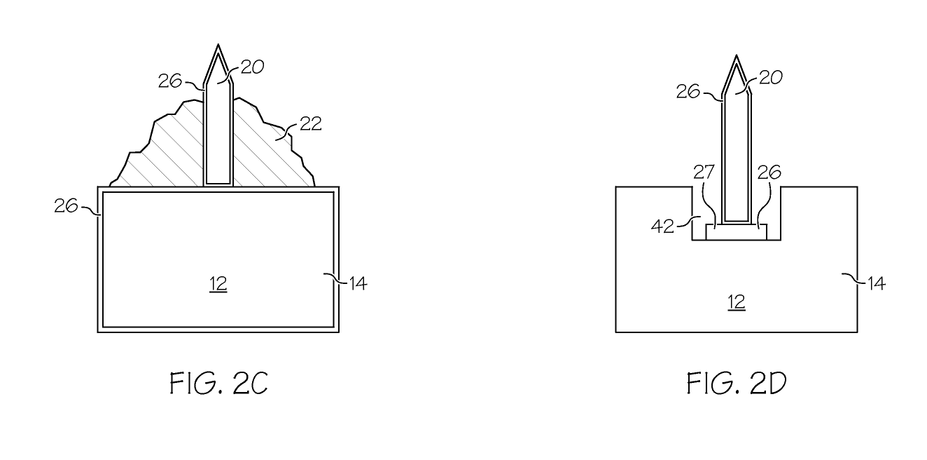

Turning to FIG. 2A, a cross-section of a valley 14 is shown therein having a radiopaque anchor 20 extending from the stent 12. As shown in FIG. 2A, the base 23 of the radiopaque anchor 20 does not directly contact the stent 12. Instead, in some embodiments, the radiopaque anchor 20 is suspended above the surface of the stent 12. More particularly, in some embodiments, the radiopaque anchor 20 is suspended in a biodegradable material 22. In some embodiments, the biodegradable material 22 comprises a polymer or gel, for example, polyvinylpyrrolidone (PVP). In some embodiments, the biodegradable material 22 comprises polyvinyl alcohol or polyesteramide.

In some embodiments, the stent 12 comprises a bio-absorbable metallic material, for example, magnesium, iron, or tungsten or alloys thereof. In some embodiments, the bio-absorbable metallic material is a magnesium alloy including combinations of magnesium, aluminum, zinc, silver, lithium, and/or rare earth metals in any desirable composition. Where the stent 12 is a metallic bio-absorbable material, in some embodiments, the radiopaque anchor 20 is separated from the stent 12 to prevent, or at least minimize, the formation of micro galvanic cells. Placement of a non-conducing material between the radiopaque anchor 20 and the stent 12 creates a barrier, minimizing or eliminating galvanic action.

In some embodiments, the biodegradable material 22 is a non-conducting material. In some embodiments, the biodegradable material 22 serves as an insulating barrier between the stent 12 and the radiopaque anchor 20. In this way, in some embodiments, the biodegradable material 22 inhibits the formation of galvanic cells due to interaction of dissimilar metals.

In addition, in some embodiments, the biodegradable material 22 provides support for the radiopaque anchor 20. By surrounding at least a portion of the radiopaque anchor 20, the biodegradable material 22 helps to prevent buckling of the radiopaque anchor 20 during deployment. In this regard, the biodegradable material 22 is soft enough to be flattened out during deployment of the stent 12, but it also has enough integrity to stabilize the radiopaque anchor 20 prior to and during deployment.

Alternatively, or in addition to the biodegradable material 22, in some embodiments, the radiopaque anchor 20 has a ceramic coating 26 over at least a portion thereof. In some embodiments, for example as shown in FIGS. 2C and 2D, the ceramic coating 26 covers the entire radiopaque anchor 20. The ceramic coating 26 can also be disposed over only a portion of the radiopaque anchor 20. In addition, in some embodiments, the stent 12 has a ceramic coating 26. In some embodiments, the ceramic coating 26 is disposed between the stent 12 and the radiopaque anchor 20. In some embodiments, the ceramic coating 26 is ultrathin, for example 15-60 nanometers; in some embodiments, the ceramic coating 26 is 15-35 nanometers in thickness, and, in some embodiments, 15-25 nanometers. Finally, in some embodiments, the ceramic coating 26 is 18 nanometers thick.

In some embodiments, the ceramic coating 26 enhances biological interaction. In some embodiments, the ceramic coating 26 comprises oxides of titanium or oxides of tantalum, which can be deposited on the surface of the stent 12 and/or radiopaque anchor 20 via physical vapor deposition (PVD) or atomic vapor deposition (AVD).

In some embodiments, the radiopaque anchor 20 is platinum, and in some embodiments is a platinum alloy, for example, an alloy of platinum and chromium. In some embodiments, the radiopaque anchor 20 is made of a platinum and cobalt alloy, for example, approximately 95% platinum and 5% cobalt. Further, in some embodiments, the radiopaque anchor 20 is made of a platinum and iridium alloy. In the context of an alloy, the term “approximately” means that percentage or ratio excludes impurities.

In some embodiments, one or more of the ceramic coatings 26 comprises an electrically non-conducting ceramic 27. As shown for example in FIG. 2D, in some embodiments, the electrically non-conducting ceramic 27 creates an insulating layer between the radiopaque anchor 20 and the stent 12, in order to prevent galvanic interaction therebetween. In particular, where the stent 12 is metallic, some embodiments employ an electrically non-conducting ceramic 27. Any desirable configuration or combination of ceramic coatings 26 and electrically non-conducting ceramics 27 can be employed, for example in layers on a portion, or the entirety, of one or both of the stent 12 and radiopaque anchor 20. In addition, any desirable configuration or combination of ceramic coatings, electrically non-conducting ceramics 27, and biodegradable materials 22 can be employed.

With further regard to FIGS. 2B and 2D, in some embodiments, the stent 12 has a cavity 42. In some embodiments, the radiopaque anchor 20 is partially disposed within the cavity 42. The cavity 42 provides additional support for the radiopaque anchor 20 during insertion and deployment of the stent 12. In some embodiments, the cavity 42 is cut via laser ablation.

In some embodiments, the stent 12 includes a plurality of cavities 42, each cavity having a radiopaque anchor 20 placed therein. In addition, it will be appreciated that the stent 12 can include some radiopaque anchors 20 which are disposed in cavities 42 and others that are located on or above the surface of the stent 12. Various combinations can be employed.

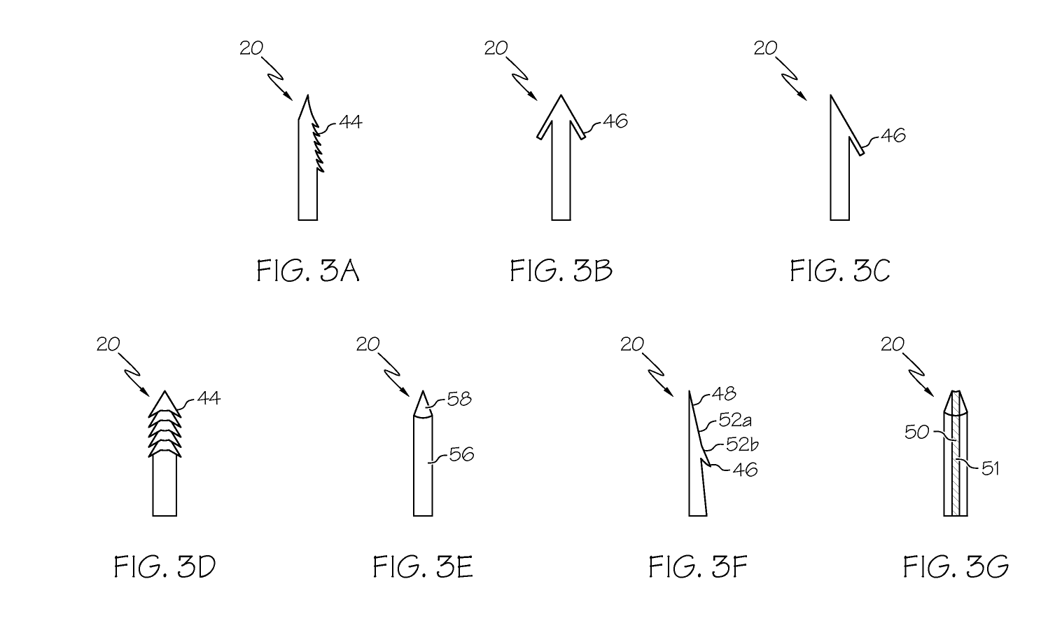

Turning now to FIGS. 3A-3G, the radiopaque anchor 20 is shown therein in a plurality of configurations. For example, FIG. 3A shows the radiopaque anchor 20 with a plurality of teeth 44 in a saw-tooth configuration. As shown in FIG. 3A, the teeth 44 do not extend entirely around the radiopaque anchor 20, but are only on one side.

FIG. 3B shows a radiopaque anchor 20 having an arrow-head configuration, with two opposing barbs 46. Alternatively, as shown in FIG. 3C, in some embodiments, the radiopaque anchor 20 comprises a single barb 46. In some embodiments, the barb 46 extends circumferentially around the radiopaque anchor 20. And, as shown in FIG. 3D, in some embodiments, the radiopaque anchor 20 comprises a plurality of teeth 44 that extend entirely around the radiopaque anchor 20.

FIG. 3E shows a radiopaque anchor 20 without any barbs, teeth, or hooks. The radiopaque anchor of FIG. 3E comprises a cylindrical portion 56 and a conical portion 58 extending from the cylindrical portion 56.

FIG. 3F shows a radiopaque anchor 20 having a single barb 46 and a pointed end 48 having two portions, 52a, 52b, of different inclination.

FIG. 3G shows a radiopaque anchor 20 having a hollow portion 50 extending along the length of the radiopaque anchor 20. In some embodiments, the hollow portion 50 extends along the entire length of the radiopaque anchor 20. In some embodiments, however, the hollow portion 50 extends along only a portion of the radiopaque anchor 20.

In some embodiments, the radiopaque anchor 20 is made of a bio-absorbable material, for example iron, magnesium, or a bio-absorbable polymer that contains an embedded amount of radiopaque nano or micro particles, for example gold nano or micro particles. In instances where the radiopaque anchor 20 is made of a bio-absorbable material, it may be designed to be absorbed after a desired period of time, for example, one, two, or three years. Additionally, in some embodiments, the bio-absorbable stent 12 is absorbed before the radiopaque anchor 20. In particular, in some embodiments involving a bio-absorbable radiopaque anchor 20 and bio-absorbable stent 12, the bio-absorbable stent 12 is absorbed less than two years after implantation and the radiopaque anchor 20 takes three or more years to be absorbed. And, in some embodiments, the bio-absorbable stent 12 is absorbed in less than one year, while the radiopaque anchor 20 takes two or more years to be absorbed. In some embodiments, the bio-absorbable stent 12 is made of magnesium, bio-absorbable polymers, amino esters and the radiopaque anchor 20 is made of iron, titanium oxide, or tungsten, and, in some embodiments. In addition, in some embodiments, the radiopaque anchor 20 is made of a porous iron material having gold nano-particles disposed within the porous iron. Also, in some embodiments, the radiopaque anchor 20 is made of a hollow iron structure having radiopaque gold nano or micro particles or other radiopaque material within the hollow cavity of the anchor 20.

In some embodiments, one or more of the radiopaque anchors 20 is filled with contrast agent 51, for example, barium sulfate or a suspension of gold nanoparticles or other contrast solution or suspension. In addition, in some embodiments, the hollow portion 50 is filled with a therapeutic agent in lieu of, or in addition to, the contrast agent 51 to offset any inflammation caused by bio-absorbable byproducts.

The radiopaque anchor 20 is not limited to the particular designs shown in FIGS. 3A-3G. Any desirable configuration or cross-section can be employed. Also, any desirable number and configuration of teeth, barbs, and so forth, can be employed.

In some embodiments, the radiopaque anchors 20 are between 10 and 100 micrometers in length and, in some embodiments, between 10 and 50 micrometers. In some embodiments, the radiopaque anchors 20 are between 5 and 25 micrometers in cross-section or diameter.

In addition, radiopaque anchors 20 can extend from any desirable portion or segment of the stent 12. In particular, in some embodiments, the radiopaque anchors 20 extend from the stent struts 16 between a peak 13 and a valley 14. Moreover, it will be appreciated that the radiopaque anchors 20 can be used in conjunction with any suitable stent design.

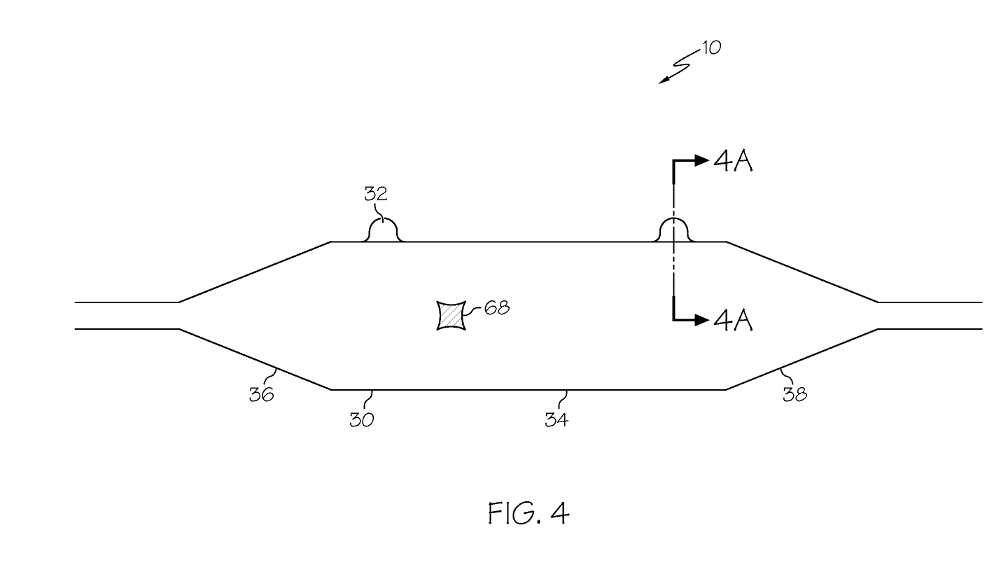

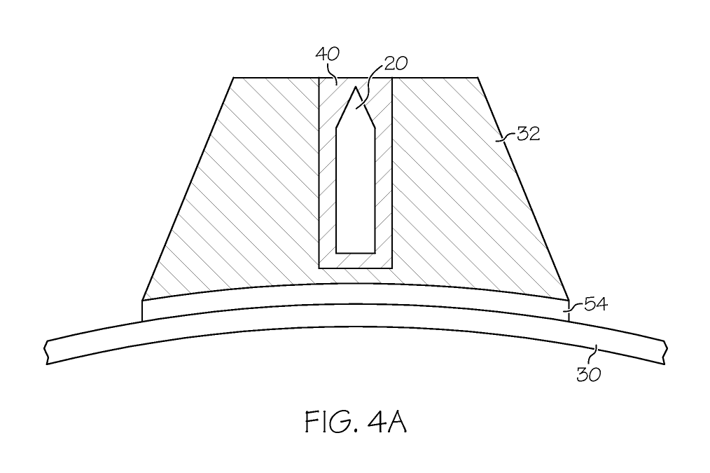

Turning now to FIG. 4, in some embodiments, the deployable medical device 10 comprises an expandable balloon 30. The balloon 30 comprises at least one housing 32 and a main body portion 34. The main body portion 34 extends between the proximal cone 36 and the distal cone 38. In some embodiments, the balloon 30 comprises a plurality of housings 32, for example, one housing 32 at or near each end of the main body portion 34. In some embodiments, the balloon 30 includes a plurality of housings 32 that are circumferentially aligned with one another on the balloon 30. In some embodiments, the balloon 30 comprises a plurality of housings 32 that are not circumferentially aligned. The housings 32 can also be arranged in a spiral or helical pattern. In some embodiments, the housings 32 are longitudinally aligned with one another. The housings 32 can be arranged in any desirable number and configuration.

With regard to FIG. 4A, the housing 32 has a radiopaque anchor 20 which is at least partially encased within the housing 32. Moreover, in some embodiments, the housing 32 has an intermediate material 40 between the radiopaque anchor 20 and the housing 32. In some embodiments, the radiopaque anchor 20 is suspended in the intermediate material 40. In some embodiments, the intermediate material 40 comprises a dissolvable material, for example sucrose, heparin, fatty acids, cholesterol, salts such as magnesium or calcium salts, or Tyrosine derived polycarbonates. The intermediate material 40 permits the radiopaque anchor 20 to be released from the balloon 30.

In addition, in some embodiments, the housing 32 comprises a polymeric material, for example silicone rubber or suitable polymer. In some embodiments, the housing 32 comprises a relatively soft, compressible material that protects the radiopaque anchor 20 during deployment, for example having a Shore hardness of 40A. In this way, the housing 32 compresses as the bio-absorbable stent 12 expands, allowing the radiopaque anchor 20 to be pressed into the adjacent tissue. In some embodiments, the housing 32 comprises a porous polymeric material, which is compressible. In addition, in some embodiments, the housing 32 comprises a hollow shell that collapses when compressed. In some embodiments, the housing 32 comprises a corrugated material that is compressible in the radial direction. Moreover, the housing 32 provides support for the radiopaque anchor 20 to prevent buckling or deformation of the radiopaque anchor 20 during insertion of the balloon 30 and deployment.

In some embodiments, the deployable medical device 10, in the form of an expandable balloon 20, has an underlayer 54. The underlayer 54 is a relatively hard, stiff material disposed between the housings 32 and the expandable balloon 30. Underlayer 54 prevents the radiopaque anchor 20 from protruding through or puncturing the expandable balloon 30. In some embodiments, the underlayer 54 is made of a polycarbonate, polyether ether ketone (PEEK), or polyethylene terephthalate (PET) material. In some embodiments, the underlayer 54 has a Rockwell hardness (R) greater than 90.

In some embodiments, the housing 32 is attached to the expandable balloon 30 for example by gluing. The housings 32 can also be attached in any suitable way.

In some embodiments, the deployable medical device 10 has a therapeutic agent 68. The term “therapeutic agent” as used herein encompasses drugs, genetic materials, biological materials, and their analogs or derivatives. The term “genetic materials” means DNA or RNA, including without limitation DNA/RNA encoding a useful protein stated below, intended to be inserted into a human body including viral vectors and non-viral vectors. A non-exclusive list of suitable therapeutic agents can be found in US Publication No. 2009/0198321, entitled “Drug-Coated Medical Devices for Differential Drug Release,” which is herein incorporated by reference.

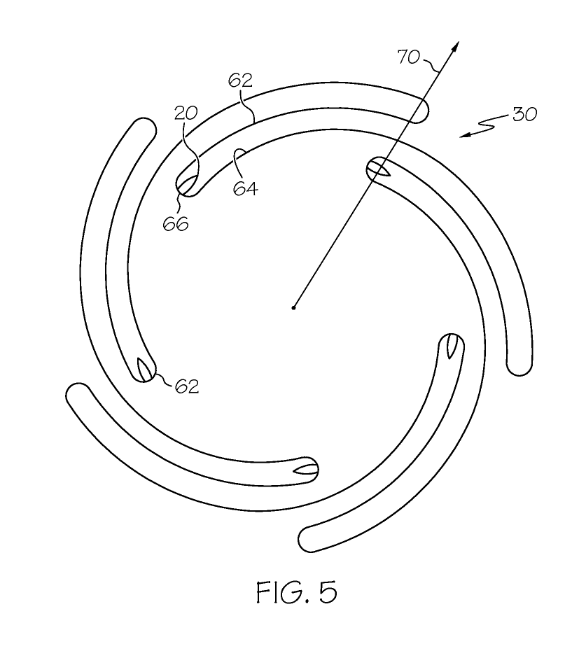

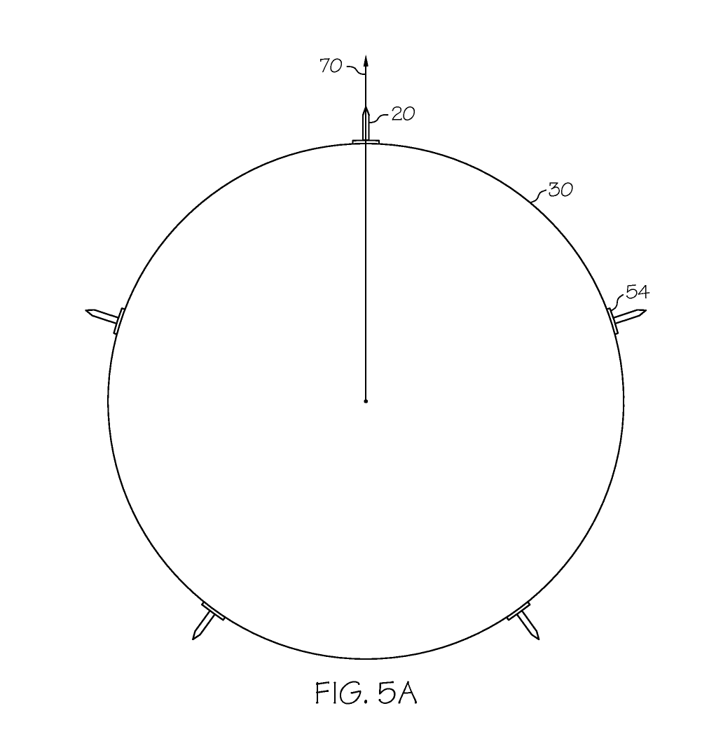

Turning to FIG. 5, in some embodiments, the radiopaque anchors 20 are disposed inside folds 62 of the expandable balloon 30 prior to expansion. More specifically, in some embodiments, the radiopaque anchors 20 are releasably attached to the expandable balloon 30, along interior portions 64 of the folds 62 such that the radiopaque anchors 20 are protected during introduction of the expandable balloon 30. In some embodiments, the radiopaque anchors 20 are placed at the base 66 of the folds 62.

As shown in FIG. 5, prior to expansion of the expandable balloon 30, the radiopaque anchors 30 are situated to lie flat within the folds 62. In some embodiments, prior to expansion of the expandable balloon 30, the longitude of the radiopaque anchors 20 is generally perpendicular to the radial direction 70 of the expandable balloon 30. In this way, the expandable balloon 30 has a reduced profile for introduction into the patient's vasculature. Upon expansion of the expandable balloon 30, as shown in FIG. 5A, however, the radiopaque anchors 20 transition from their introduction configuration to a deployed configuration. In the deployed configuration, the longitude of the radiopaque anchors 20 is oriented generally parallel to the radial direction 70 of the expandable balloon 30.

Subsequently, as the expandable balloon 30 continues to expand, the radiopaque anchors 20 are pressed into the adjacent tissue. Thereafter, in some embodiments, the radiopaque anchors 20 detach from the expandable balloon 30, and, upon deflation of the expandable balloon 30, the radiopaque anchors 20 remain in the patient.

Although shown specifically with a stent and balloon, the radiopaque anchors 20 can be used with any suitable medical device. In addition, the radiopaque anchors 20 are not limited to use in the particular types of balloons or stents depicted in the attached figures.

In some embodiments, the radiopaque anchors 20 are cast in a mold. Other ways of forming the radiopaque anchors 20 are also permissible.

The radiopaque anchors 20 are inserted into the patient's tissue in a radial fashion. In this way, the possibility that the radiopaque anchors 20 will detach from the vessel wall, for example, at a later time, is greatly reduced when compared to conventional marker construction used in combination with known deployable medical devices.

In some embodiments, a balloon 30 having at least one radiopaque anchor 20 thereon is used in combination with a stent 12. The at least one radiopaque anchor 20 is placed on the balloon 30 either distally, proximally, or both, of the respective ends of the stent 12. Consequently, as the balloon 30 is expanded, the at least one radiopaque anchor 20 is deployed in conjunction with the stent 12.

Although particular features are shown or described with respect to particular embodiments disclosed herein, it will be appreciated that these features can be combined with the features or substituted for the features of other embodiments.

The above disclosure is intended to be illustrative and not exhaustive. This description will suggest many variations and alternatives to one of ordinary skill in this field of art. All these alternatives and variations are intended to be included within the scope of the claims where the term “comprising” means “including, but not limited to.” Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the preferred and alternate embodiments of the invention. Those skilled in the art may recognize other equivalents to the specific embodiment described herein which equivalents are intended to be encompassed by the claims attached hereto.

What is claimed is:

1. An implantable medical device comprising:
a bio-absorbable stent;
at least one radiopaque anchor extending radially from the bio-absorbable stent and being attached thereto, the at least one radiopaque anchor being made of a different material than the bio-absorbable stent; and
a biocompatible polymer contacting at least a portion of the bio-absorbable stent and at least a portion of the at least one radiopaque anchor.

2. The implantable medical device of claim 1, wherein the at least one radiopaque anchor comprises at least one barb.

3. The implantable medical device of claim 1, wherein the biocompatible polymer surrounds the at least one radiopaque anchor.

4. The implantable medical device of claim 1, wherein the bio-absorbable stent is polymeric.

5. The implantable medical device of claim 1, wherein the bio-absorbable stent is metallic.

6. The implantable medical device of claim 1, wherein the at least one radiopaque anchor comprises a pin.

7. The implantable medical device of claim 6, wherein the pin has a circular cross-section and a diameter of 10 microns.

8. The implantable medical device of claim 1 further comprising a ceramic coating disposed on at least a portion of the at least one radiopaque anchor.

9. The implantable medical device 8, wherein the ceramic coating is selected from the group consisting of titanium oxides and tantalum oxides.

10. The implantable medical device of claim 8, wherein the ceramic coating is between 10 and 30 nanometers thick.

11. The implantable medical device of claim 1, wherein the radiopaque anchor is made of an alloy selected from the group consisting of platinum, cobalt, iridium, chromium, and combinations thereof.

12. The implantable medical device of claim 11, wherein the alloy is approximately 95% platinum and 5% cobalt.

13. A stent comprising:
a bio-absorbable framework; and
a plurality of radiopaque anchors extending radially from the bio-absorbable framework and being attached thereto, the radiopaque anchors having appointed end and at least one barb, wherein the radiopaque anchors are made of a different material than the bio-absorbable stent.

14. The stent of claim 13, wherein the radiopaque anchors are not bio-absorbable.

15. The stent of claim 13, wherein the radiopaque anchors have a length and at least one of the radiopaque anchors is hollow along its length.

16. A medical balloon comprising:
an unexpanded configuration and an expanded configuration;
at least one radiopaque anchor extending radially from the medical balloon when the medical balloon is in the expanded configuration, the at least one radiopaque anchor being detachably fastened to the medical balloon.

17. The medical balloon of claim 16, wherein the medical balloon comprises a plurality of folds in the unexpanded configuration, the at least one radiopaque marker disposed within one of the folds.

18. The medical balloon of claim 16 further comprising a biocompatible polymer between at least a portion of the radiopaque anchor and the medical balloon.
